(19) United States
(12) Patent Application Publication
(10) Pub. No.: US 2010/0137650 A1
(43) Pub. Date: Jun. 3, 2010

(54) SHELL CATALYST, PROCESS FOR ITS PREPARATION AND USE

(75) Inventors: Hans-Jörg Wölk, Rosenheim (DE); Alfred Hagemeyer, Bad Aibling (DE); Frank Grossmann, Munchen (DE); Silvia Neumann, Grosskarolinenfeld (DE); Gerhard Mestl, Munchen (DE)

Correspondence Address: CONNOLLY BOVE LODGE & HUTZ LLP, 1875 EYE STREET, N.W., SUITE 1100, WASHINGTON, DC, 20006, US

(73) Assignee: Sud-Chemie AG, Munchen (DE)

(21) Appl. No.: 12/625,085

(22) Filed: Nov. 24, 2009

(30) Foreign Application Priority Data
Nov. 25, 2008 (DE) 10 2008 058 971.3

(51) Int. Cl. B01J 23/755; B01J 21/16; B01J 23/72; B01J 23/34; C07C 5/10; C07C 5/03; C07C 29/14

(52) U.S. Cl. 568/700; 502/74; 585/270; 585/276

(57) ABSTRACT

The present invention relates to a shell catalyst, a process for its preparation and also the use of the shell catalyst according to the invention.

The present invention relates to a shell catalyst.

The hydrogenation of aromatics, olefins, alkines and oxo products is carried out in chemical engineering predominantly using supported Ni catalysts. As a rule, these catalysts comprise an open-pored catalyst support which is thoroughly loaded with Ni.

The Ni catalysts of the state of the art have a relatively low activity.

The object of the present invention is therefore to provide an Ni catalyst which has a relatively high activity.

According to the invention, this object is achieved by a shell catalyst comprising an open-pored catalyst support with a shell in which Ni and Cu and/or Pd and also in addition Mn and/or Mo are contained or in which Ni and also Mn and Mo are contained.

Surprisingly, it was discovered that shell catalysts which comprise an open-pored catalyst support with a shell in which Ni, Cu and Mn; Ni, Cu and Mo; Ni, Pd and Mn; Ni, Pd and Mo; Ni, Cu, Pd and Mn; Ni, Cu, Pd and Mo; Ni, Cu, Pd, Mn and Mo; Ni, Cu, Mn and Mo; Ni, Pd, Mn and Mo; or Ni, Mn and Mo are contained have a relatively high activity.

In addition, it has been established that the shell catalyst according to the invention is characterized by a relatively high selectivity, in particular in hydrogenation reactions.

Shell catalysts are known in the state of the art. A distinction is drawn in the case of shell catalysts between “egg-shell” and “egg-white” shell catalysts. An “egg-shell” catalyst is a shell catalyst in which the catalytically active substance is present in an outer shell of the catalyst support, wherein the shell of the outer surface of the support extends inwardly and the core of the catalyst support is free of catalytically active substance. On the other hand, in an “egg-white” shell catalyst an inner shell is loaded with the catalytically active substance in a zone of the catalyst support close to the surface, roughly beneath the outer support surface, wherein the outer shell not occupied by catalytically active substance is meant to trap catalyst poisons and thus protect the catalytically active substance beneath it from poisoning. Also in the case of shell catalysts of the “egg-white” type the core of the catalyst support is free of catalytically active substance.

The shell catalyst according to the invention is a shell catalyst of the “egg-shell” or “egg-white” type, preferably a shell catalyst of the “egg-shell” type.

According to a preferred embodiment of the catalyst according to the invention, it is provided that Ni and also Cu and/or Pd are present in the oxidation state 0.

If the catalyst according to the invention is present in its active form, then Ni and Cu; Ni and Pd; Ni, Cu and Pd; or only Ni (in the case of an Ni/Mn/Mo shell catalyst) have the oxidation state 0. Otherwise, the named metals can be present in any oxidation state and in the form of any chemical compound from which the metal components can be converted into the oxidation state 0.

According to a further preferred embodiment of the present invention, it is provided that Mn and/or Mo are present in oxide form.

If the catalyst according to the invention is present in its active form, then Mn, Mo or Mn and Mo are present in oxide form. In non-active form the metals can be present in the form of any chemical compound in the shell from which they can be converted into the oxide form.

In a particularly preferred embodiment of the present invention, Ni, Cu and Mn are contained in the shell of the catalyst support.

If Ni, Cu and Mn are contained in the shell of the catalyst support, it is provided according to a further preferred embodiment of the shell catalyst according to the invention that the Ni/Cu atomic ratio of the shell catalyst is 0.5 to 30, preferably 1 to 20 and more preferably 2 to 10 and independently thereof the Ni/Mn atomic ratio is 1 to 100, preferably 1 to 50, more preferably 2 to 30 and most preferably 3 to 20.

According to an alternative embodiment of the shell catalyst according to the invention, the shell of the catalyst support contains Ni, Pd and Mo.

If Ni, Pd and Mo are contained in the shell of the catalyst support, it is provided according to a preferred embodiment of the shell catalyst according to the invention that the Ni/Pd atomic ratio of the shell catalyst is 10 to 500, preferably 20 to 400 and more preferably 30 to 300 and independently thereof the Ni/Mo atomic ratio is 1 to 100, preferably 1 to 50, more preferably 1 to 30 and most preferably 2 to 20.

If Ni, Mn and Mo are contained in the shell of the catalyst support, it is provided according to a preferred embodiment the shell catalyst according to the invention that the Ni/Mn atomic ratio is 1 to 100, preferably 1 to 50, more preferably 2 to 30 and most preferably 3 to 20 and independently thereof the Ni/Mo atomic ratio is 1 to 100, preferably 1 to 50, more preferably 1 to 30 and most preferably 2 to 20.

According to another preferred embodiment of the shell catalyst according to the invention, it is provided that the proportion of Ni in the shell catalyst is 5 wt.-% to 15 wt.-% relative to the weight of the shell catalyst.

The smaller the thickness of the shell, the higher the selectivity of the shell catalyst according to the invention. According to a further preferred embodiment of the catalyst according to the invention, the shell of the catalyst has a thickness of less than 2200 μm, preferably less than 1000 μm, preferably less than 500 μm and further preferably 30 to 200 μm. The thickness of the shell of the catalyst can be measured visually by means of a microscope.

It has been established that, the smaller the specific surface area of the catalyst support, the higher the selectivity of the catalyst according to the invention. In addition, the smaller the specific surface area of the catalyst support, the greater the chosen thickness of the shell can be without having to accept appreciable losses in selectivity. According to a preferred embodiment of the catalyst according to the invention, it is preferred that the specific surface area of the catalyst support is less than/equal to 160 m²/g, preferably less than 140 m²/g, preferably less than 135 m²/g, further preferably less than 120 m²/g, more preferably less than 100 m²/g, still more preferably less than 80 m²/g and particularly preferably less than 65 m²/g. By “specific surface area” of the catalyst support is meant the BET surface area of the catalyst support which is determined by means of nitrogen adsorption according to DIN 66131 and DIN 66132. A publication of the BET method is to be found J. Am. Chem. Soc. 60, 309 (1938).

According to a further preferred embodiment of the catalyst according to the invention, it can be provided that the catalyst support has a specific surface area of 160 to 40 m²/g, preferably between 140 and 50 m²/g, preferably between 135 and 50 m²/g, further preferably between 120 and 50 m²/g, more preferably between 100 and 50 m²/g and most preferably between 100 and 60 m²/g.

It was found that the selectivity of the catalyst according to the invention depends on the integral pore volume of the catalyst support. According to a further preferred embodiment of the catalyst according to the invention, the catalyst support has an integral pore volume according to BJH of more than/equal to 0.30 ml/g, preferably more than 0.35 ml/g and preferably more than 0.40 ml/g. The integral pore volume of the catalyst support is determined according to DIN 66134 (determination of the pore-size distribution and of the specific surface area of mesoporous solids by nitrogen sorption (process according to Barrett, Joyner and Halenda (BJH)).

According to a further preferred embodiment of the present invention, it is preferred that the catalyst support has an integral pore volume according to BJH of 0.3 ml/g to 1.2 ml/g, preferably 0.4 ml/g to 1.1 ml/g and more preferably 0.5 ml/g to 1.0 ml/g.

To determine the specific surface area and the integral pore volume of the catalyst support, the sample is preferably measured with a fully automatic nitrogen porosimeter from Mikromeritics, type ASAP 2010, by means of which an adsorption and also desorption isotherm can be recorded.

It can be preferred according to a further preferred embodiment of the catalyst according to the invention that at least 80%, preferably at least 85% and by preference at least 90%, of the integral pore volume of the catalyst support is formed from mesopores and macropores. This counteracts a reduced activity, effected by diffusion limitation, of the catalyst according to the invention, in particular of shells with a relatively large shell thickness. By micropores, mesopores and macropores are meant in this case pores which have a diameter of less than 2 nm, a diameter of 2 to 50 nm and a diameter of more than 50 nm respectively. The proportion of mesopores and macropores in the integral pore volume is obtained from the pore-size distribution which is determined according to DIN 66134 (determination of the pore-size distribution and of the specific surface area of mesoporous solids by nitrogen sorption (process according to Barrett, Joyner and Halenda (BJH)).

According to a further preferred embodiment of the present invention, the catalyst support of the catalyst according to the invention can have a bulk density of more than 0.30 g/ml, preferably more than 0.35 g/ml and particularly preferably a bulk density of between 0.35 and 0.6 g/ml.

In order to further reduce the pore diffusion limitation, it can be provided according to a further preferred embodiment of the catalyst according to the invention that the catalyst support has an average pore diameter of 8 nm to 50 nm, preferably of 10 nm to 35 nm and preferably of 11 nm to 30 nm. The average pore diameter is obtained from the pore-size distribution, to be ascertained as indicated above, of the catalyst support.

According to a further preferred embodiment of the catalyst according to the invention, the catalyst support has an acidity of between 1 and 150 μval/g, preferably between 5 and 130 μval/g, preferably between 10 and 100 μval/g and particularly preferably between 10 and 60 μval/g. The acidity of the support can be influenced for example by the choice of support material or by an impregnation of the catalyst support or catalyst with acid.

The acidity of the catalyst support is determined as follows: 100 ml water (with a pH blank value) is added to 1 g of the finely ground catalyst support and extraction carried out for 15 minutes accompanied by stirring. Titration to at least pH 7.0 with 0.01 n NaOH solution follows, wherein the titration is carried out in stages; 1 ml of the NaOH solution is firstly added dropwise to the extract (1 drop/second), followed by a 2-minute wait, the pH is read, a further 1 ml NaOH added dropwise, etc. The blank value of the water used is determined and the acidity calculation corrected accordingly. The titration curve (ml 0.01 NaOH against pH) is then plotted and the intersection point of the titration curve at pH 7 determined. The mole equivalents which result from the NaOH consumption for the intersection point at pH 7 are calculated in 10⁻⁶ equiv/g catalyst support.

$$Total acid \text{:} \frac{10 * ml 0.01 n NaOH}{1 support} = µ val \text{/} g$$

According to a preferred embodiment, the catalyst support of the catalyst according to the invention is formed as a shaped body.

In principle, the catalyst support of the catalyst according to the invention can have any shape. However, it is preferred if the catalyst support is formed as a sphere, cylinder (also with rounded end surfaces), perforated cylinder (also with rounded end surfaces), trilobe, “capped tablet”, tetralobe, ring, doughnut, star, cartwheel, “reverse” cartwheel, or as a strand, preferably as a ribbed strand or star strand, particularly preferably as a sphere.

The catalyst support preferably measures at most 1 mm to 50 mm, preferably 2 mm to 15 mm.

If the catalyst support is formed as a sphere, then the catalyst support preferably has a diameter of 1 mm to 25 mm, preferably a diameter of 3 mm to 10 mm.

According to a further preferred embodiment of the catalyst according to the invention, the catalyst support consists at least 50 wt.-%, preferably 80 wt.-% and particularly preferably at least 90 wt.-% of SiO₂, Al₂O₃, an aluminium silicate, ZrO₂, TiO₂, HfO₂, MgO, niobium oxide or a natural sheet silicate or at least 50 wt.-%, preferably at least 80 wt.-% and particularly preferably at least 90 wt.-% of a mixture of two or more of the abovenamed materials.

According to a further preferred embodiment of the shell catalyst according to the invention, it is provided that the catalyst support consists at least 80 wt.-%, preferably at least 90 wt.-%, of a natural sheet silicate.

According to a further preferred embodiment of the catalyst according to the invention, it is provided that the catalyst support consists at least 80 wt.-% of montmorilionite.

By “natural sheet silicate”, for which “phyllosilicate” is also used in the literature, is meant within the framework of the present invention treated or untreated silicate material from natural sources, in which SiO₄ tetrahedra, which form the structural base unit of all silicates, are cross-linked with each other in layers of the general formula [Si₂O5]²⁻. These tetrahedron layers alternate with so-called octahedron layers in which a cation, principally Al and Mg, is octahedrally surrounded by OH or O. A distinction is drawn for example between two-layer phyllosilicates and three-layer phyllosilicates. Sheet silicates preferred within the framework of the present invention are clay minerals, in particular kaolinite, beidellite, hectorite, saponite, nontronite, mica, vermiculite and smectites, wherein smectites and in particular montmorillonite are particularly preferred. Definitions of the term “sheet silicates” are to be found for example in “Lehrbuch der anorganischen Chemie”, Hollemann Wiberg, de Gruyter, 102^{nd} edition, 2007 (ISBN 978-3-11-017770-1) or in “Römpp Lexikon Chemie”, 10^{th} edition, Georg Thieme Verlag under “Phyllosilikat”. Typical treatments to which a natural sheet silicate is subjected before use as support material include for example a treatment with acids, whereby acid-activated sheet silicate or a bleaching earth is obtained, and/or calcining. A natural sheet silicate particularly preferred within the framework of the present invention is a bentonite. Admittedly, bentonites are not really natural sheet silicates, more a mixture of predominantly clay minerals containing sheet silicates, predominantly montmorillonite. Thus in the present case, where the natural sheet silicate is a bentonite, it is to be understood that the natural sheet silicate is present in the catalyst support in the form of or as a constituent of a bentonite.

The catalyst according to the invention is usually prepared by subjecting a plurality of catalyst supports to a “batch” process during the individual process steps of which the catalyst supports are for example subjected to relatively high mechanical load stresses communicated by stirring and mixing tools. In addition, the catalyst according to the invention can be subjected to a strong mechanical load stress during the filling of a reactor, which can result in an undesired formation of dust and damage to the catalyst support, in particular to its shell containing the metals. The catalyst according to the invention therefore preferably has a hardness greater than/equal to 20 N, preferably greater than/equal to 30 N, further preferably greater than/equal to 40 N and most preferably greater than/equal to 50 N.

The hardness is ascertained by means of an 8M tablet-hardness testing machine from Dr. Schieuniger Pharmatron AG, determining the average for 99 shell catalysts after drying of the catalyst at 130° C. for 2 h, wherein the apparatus settings are as follows:
Hardness: N
Distance from the catalyst support: 5.00 mm
Time delay: 0.80 s
Feed type: 6 D
Speed: 0.60 mm/s

The hardness of the catalyst can be influenced for example by varying certain parameters of the process for the preparation of the catalyst support, for example through the selection of the raw materials, the calcining duration and/or the calcining temperature of an uncured catalyst support formed from the corresponding support mixture, or by particular loading materials, such as for example methyl cellulose or magnesium stearate.

It can be provided according to a further preferred embodiment of the catalyst according to the invention that the water absorbency of the catalyst support is 40% to 75%, preferably 50% to 70% calculated as the weight increase due to water absorption. The absorbency is determined by steeping 10 g of the support sample in deionized water for 30 min until gas bubbles no longer escape from the support sample. The excess water is then decanted and the steeped sample blotted with a cotton towel to remove adhering moisture from the sample. The water-laden catalyst support is then weighed and the absorbency calculated as follows:

(amount weighed out (g)−amount weighed in (g))×10=water absorbency (%)

According to a further preferred embodiment of the catalyst according to the invention, it is provided that the catalyst has at least one promoter selected from the group consisting of P, Na, K, Co and Mg. The promoter can in principle be present in any chemical form which is known to a person skilled in the art to be suitable for the purpose according to the invention. However, it is preferred according to the invention if the promoter is present in the catalyst in elemental or oxide form.

The present invention furthermore relates to a process, in particular a process for the preparation of a shell catalyst according to the invention, wherein in the process a catalyst support is sprayed with a solution in which an Ni compound and also a Cu compound and/or a Pd compound and also furthermore an Mn compound and/or an Mo compound are contained dissolved or in which an Ni compound and also an Mn compound and an Mo compound are contained dissolved.

It has been established that shell catalysts prepared according to the process according to the invention have a relatively thin shell with a relatively uniform thickness, which leads to a relatively high selectivity of the catalyst.

According to a preferred embodiment of the process according to the invention, it is provided that at least one chemical compound of at least one element selected from the group consisting of P, Na, K, Co and Mg is contained dissolved in the solution.

According to a preferred embodiment of the process according to the invention, it is provided that the process further comprises:
the production of a fluidized bed of catalyst supports by means of a gas, wherein the catalyst supports move in the fluidized bed on an elliptical or toroidal path;
the spraying of the catalyst support moving in the fluidized bed on an elliptical or toroidal path with the solution;

The process according to the invention is preferably carried out by producing a fluidized bed in which the catalyst supports move on an elliptical or toroidal path or, in other words, in which the catalyst supports circulate elliptically or toroidally.

In the state of the art, the transition of the particles of a bed into a state in which the particles can move completely freely (fluidized bed) is called the loosening point (incipient fluidization point) and the corresponding fluidization velocity is called the loosening velocity. According to the invention it is preferred that in the process according to the invention the fluidization velocity (of the gas) is up to 4 times the loosening velocity, preferably up to 3 times the loosening velocity and more preferably up to 2 times the loosening velocity.

According to an alternative embodiment of the process according to the invention, it can be provided that the fluidization velocity is up to 1.4 times the common logarithm of the loosening velocity, preferably up to 1.3 times the common logarithm of the loosening velocity and more preferably up to 1.2 times the common logarithm of the loosening velocity.

Unlike when operating in a conventional fluid bed, the effect of the combined action of the spraying with the fluidized-bed-like elliptical or toroidal circulating movement of the catalyst supports in the fluidized bed is that the individual catalyst supports pass through the spray nozzle at an approximately identical frequency. In addition, the circulation process also sees to it that the individual catalyst supports rotate about their own axis, for which reason the catalyst supports are impregnated particularly evenly with solution.

In the process according to the invention the catalyst supports preferably circulate elliptically or toroidally in the fluidized bed. However, it is particularly preferred that the catalyst supports move in the fluidized bed on a toroidal path.

To give an idea of how the catalyst supports move in the fluidized bed, it may be stated that in the case of “elliptical circulation” the catalyst supports move in the fluidized bed in a vertical plane on an elliptical path, the size of the major and minor axes varying little. In the case of “toroidal circulation” the catalyst supports move in the fluidized bed in a vertical plane on an elliptical path, the size of the major and minor axes varying little, and in the horizontal plane on a circular path, the size of the radius varying. On average, the catalyst supports move in the case of “elliptical circulation” in a vertical plane on an elliptical path, in the case of “toroidal circulation” on a toroidal path, i.e. a catalyst support covers the surface of a torus helically with vertical elliptical section.

In a further preferred embodiment, the process according to the invention furthermore comprises
the drying of the catalyst supports sprayed with the solution.

Within the framework of the process according to the invention, the catalyst supports sprayed with the solution are preferably dried continuously by means of the gas to produce the fluidized bed. However, it can also be provided that a separate drying step is carried out after spray impregnation with continuous drying or without drying. In the first case, for example, the drying speed and with it for example the thickness of the shell can be set by the temperature of the gas or of the catalyst supports, in the second case the drying can be carried out using any drying method known to a person skilled in the art to be suitable.

It is preferred according to the invention that the drying takes place at a temperature of 20° C. to 200° C., preferably between 40° C. and 150° C. and particularly preferably between 70° C. and 120° C., wherein the drying can take place both at normal pressure and in a vacuum.

According to a further preferred embodiment, the process according to the invention furthermore comprises
the calcining of the catalyst supports sprayed with the solution at a temperature at which the metal component of the metal compounds sprayed onto the catalyst supports is converted into an oxide form.

As a result of the calcining, firstly, the metal components are fixed to the catalyst support, and, secondly, the metals Ni, Cu and Pd can be converted relatively easily into the metal state from the oxide form.

Within the framework of the present invention, the calcining can for example be carried out in a temperature range of 200° C. to 1000° C., preferably in a temperature range of 300° C. to 800° C., further preferably in a temperature range of 350° C. to 750° C. and particularly preferably in a temperature range of 400° C. to 500° C.

The duration of the calcining normally lie in the range of 1 min to 48 h, preferably in a range of 30 min to 12 h and more preferably in a range of 1 h to 7 h, wherein a calcining duration of 2 h to 5 h is particularly preferred.

According to the present invention, the catalyst support sprayed with the metal compound can preferably be calcined under a protective gas if the metal compounds decompose, e.g. by autoreduction, to metal of the oxidation state 0. A separate reduction step can thereby be avoided.

According to the present invention, it is particularly preferred that the protective gas is a gas selected from the group consisting of the noble gases, CO₂, nitrogen and mixtures of two or more of the above-named. By protective gas is meant gases or gas mixtures which can be used as an inert protective atmosphere, for example to avoid unwanted chemical reactions. Within the framework of the present invention, the noble gases helium, neon, argon, krypton or xenon in particular, or mixtures of two or more of the above-named, can be used as protective gas, wherein argon is particularly preferred as protective gas. Besides the noble gases or in addition to them, nitrogen for example can also be used as protective gas. A protective-gas atmosphere particularly preferred according to the process of the present invention comprises the noble gas argon and also nitrogen.

According to a further preferred embodiment, the process according to the invention furthermore comprises
the conversion of the metal component of the metal compounds sprayed onto the catalyst support to the oxidation state 0. In the present case, by metal compounds are meant those of Ni, Cu and Pd and not those of Mo or Mn.

According to the present invention, it is preferred that the conversion of the metal component of the metal compounds to the oxidation state 0 takes place by means of a reducing agent.

Gaseous or vaporable reducing agents such as for example H₂, CO, NH₃, formaldehyde, methanol and hydrocarbons are preferably used, wherein the gaseous reducing agents can also be diluted with inert gas, such as for example carbon dioxide, nitrogen or argon. An inert gas-diluted reducing agent is preferably used. Mixtures of hydrogen with nitrogen or argon, preferably with a hydrogen content between 1 vol.-% and 50 vol.-%, are preferred.

The quantity of reducing agent is preferably chosen such that during the treatment period at least the equivalent required for complete conversion of the metals is passed over the catalyst. Preferably, however, an excess of reducing agent is passed over the catalyst in order to ensure a rapid and complete conversion.

Preferably, the metal is converted into the oxidation state 0 pressureless, i.e. at an absolute pressure of approx. 1 bar. For the preparation of industrial quantities of catalyst according to the invention a rotary tube oven or fluid-bed reactor is preferably used in order to ensure an even reduction.

According to the present invention, the metals are preferably converted into the oxidation state 0 at a temperature of 100° C. to 500° C.

The metals can in principle be converted into the oxidation state 0 at any temperature which is known to a person skilled in the art to be suitable for the purpose according to the invention. Within the framework of the present invention, the metals can be converted into the oxidation state 0 in a temperature range of 100° C. to 500° C., preferably in a temperature range of 200° C. to 500° C., and more preferably in a temperature range of 300° C. to 450° C.

The conversion of Ni and Cu, of Ni and Pd or of Ni, Cu and PD can also be carried out in situ, i.e. in the process reactor, or else ex situ, i.e. in a special reduction reactor. Conversion ex situ can be carried out for example with 5 vol.-% hydrogen in nitrogen, for example by means of forming gas, at temperatures in the range of preferably 150° C. to 500° C. over a period of 5 hours.

In a further preferred embodiment of the process according to the invention, an Ni compound, a Cu compound and an Mn compound are contained dissolved in the solution. This solution is free of Pd and Mo.

In a further preferred embodiment of the process according to the invention, an Ni compound, a Pd compound and an Mo compound are contained dissolved in the solution. This solution is free of Cu and Mo.

In a further preferred embodiment of the process according to the invention, the metal compounds contained in the solution are halogen-free metal compounds, preferably nitrate compounds. An Mo nitrate is not known and is therefore preferably used in the solution as Mo compound pre-dissolved in nitric acid or phosphoric acid or water.

The metal compounds to be used in the process according to the invention are preferably halogen-free as halogens act as catalyst poisons for a large number of catalytically active metals and accordingly can lead to a deactivation of the catalyst to be prepared.

In a further preferred embodiment of the process according to the invention, the solution is an aqueous solution.

In a particularly preferred embodiment the process according to the invention is carried out using a device which is set up to produce a fluidized bed of a particulate material by means of a gas, wherein the particles of the material move in the fluidized bed on an elliptical or toroidal path. Such devices are described for example in WO 2006/027009 A1, DE 102 48 116 B3, EP 0 370 167 A1, EP 0 436 787 B1, DE 199 04 147 A1, DE 20 2005 003 791 U1, the contents of which are incorporated in the present invention through reference.

Devices which are particularly preferred according to the invention are sold by Innojet Technologies under the names Innojet® Ventilus or Innojet® AirCoater. These devices comprise a cylindrical container with a fixedly and immovably installed container bottom in the centre of which a spraying nozzle is mounted. The bottom consists of circular plates arranged in steps above each other. The process air flows horizontally into the container between the individual plates eccentrically, with a circumferential flow component, outwardly towards the container wall. So-called air flow beds form on which the catalyst supports are first transported outwardly towards the container wall. A perpendicularly oriented process air stream which deflects the catalyst supports upwards is deflected outside along the container wall. Having reached the top, the catalyst supports move on a more or less tangential path back towards the centre of the bottom in the course of which they pass through the spray mist of the nozzle. After passing through the spray mist, the described movement process begins again. The described process-air guiding provides the basis for a largely homogeneous, toroidal fluidized-bed-like circulating movement of the catalyst supports.

To produce a catalyst support fluidized bed in which the catalyst supports circulate elliptically or toroidally in a manner that is simple in terms of process engineering, and thus inexpensive, it is provided according to a further preferred embodiment of the process according to the invention that the device comprises a process chamber with a bottom and a side wall, wherein the gas is fed into the process chamber with a horizontal movement component aligned radially outwards through the bottom of the process chamber which preferably constructed of several overlapping annular guide plates laid over one another between which annular slots are formed, in order to produce the catalyst support fluidized bed.

Because gas is fed into the process chamber with a horizontal movement component aligned radially outwards, an elliptical circulation of the catalyst supports in the fluidized bed is brought about. If the structures are to circulate toroidally in the fluidized bed, the catalyst supports must also be subjected to a further circumferential movement component which forces the supports onto a circular path.

The process of the present invention therefore includes, according to a preferred embodiment, the feature that the gas fed into the process chamber is subjected to a circumferential flow component.

The circumferential movement component can be imposed on the catalyst supports for example by attaching suitably aligned guide rails to the side wall to deflect the catalyst supports.

According to a preferred embodiment of the process according to the invention, however, it is provided that a circumferential flow component is imposed on the gas fed into the process chamber. The production of the fluidized bed in which the catalyst supports circulate toroidally is thereby ensured in a simple manner in terms of process engineering.

To subject the gas fed into the process chamber to the circumferential flow component, it can be provided according to a further preferred embodiment of the process according to the invention that suitably shaped and aligned gas guide elements are arranged between the annular guide plates. As an alternative or in addition to this, it can be provided that the gas fed into the process chamber is subjected to the circumferential flow component by feeding additional gas, with a movement component aligned diagonally upwards, into the process chamber through the bottom of the process chamber, preferably in the area of the side wall of the process chamber.

it can be provided that the structures circulating in the fluidized bed are sprayed with the solution by means of an annular gap nozzle which sprays a spray cloud, wherein the plane of symmetry of the spray cloud runs parallel or substantially parallel to the plane of the device bottom. Due to the 360° circumference of the spray cloud, the catalyst supports moving downwards in the middle can be sprayed particularly evenly with the solution. The annular gap nozzle, i.e. its mouth, is preferably completely embedded in the fluidized bed.

According to a further preferred embodiment of the process according to the invention, it is provided that the annular gap nozzle is arranged in the middle in the bottom of the container and the mouth of the annular gap nozzle is embedded in the fluidized bed. It is thereby ensured that the distance covered by the drops of the spray cloud until they meet a catalyst support is relatively short and, accordingly, relatively little time remains for the drops to coalesce into larger drops, which could work against the formation of a largely uniform shell thickness.

According to a further preferred embodiment of the process according to the invention, it can be provided that a gas support cushion is produced on the underside of the spray cloud. The bottom cushion keeps the bottom surface largely free of sprayed solution, which means that almost all of the sprayed solution is introduced into the fluidized bed, with the result that hardly any spray losses occur.

According to a further preferred embodiment of the process according to the invention, it is provided that the gas for the production of the fluidized bed is selected from the group consisting of air, oxygen, nitrogen and the noble gases and also mixtures of the above gases.

According to a further embodiment of the process according to the invention, it is preferred that the process, i.e. the spraying of the catalyst supports with the solution, is carried out at a temperature greater than/equal to 60° C., preferably at a temperature greater than/equal to 70° C., preferably at a temperature greater than/equal to 80° C. and most preferably at a temperature greater than/equal to 90° C. to 120° C.

To prevent drops of the spray cloud from drying prematurely, it can be provided that the gas is enriched, before being fed into the device, with the solvent of the solution to be used, preferably in a range of 10% to 50% of the saturation vapour pressure (at the process temperature). The solvent added to the gas and also solvents from the drying of the catalyst supports can be separated from the gas by means of suitable cooling aggregates, condensers and separators and returned to the solvent enricher by means of a pump.

The present invention furthermore relates to the use of a shell catalyst according to the invention for the hydrogenation of aromatics, alkines, olefins, aldehydes and oxo products, in particular for the hydrogenation of 2-ethyl hexenal or butynediol.

The following description of a preferred device for carrying out the process according to the invention and also the description of movement paths of catalyst supports serve, in connection with the drawing, to explain the invention. There are shown in:

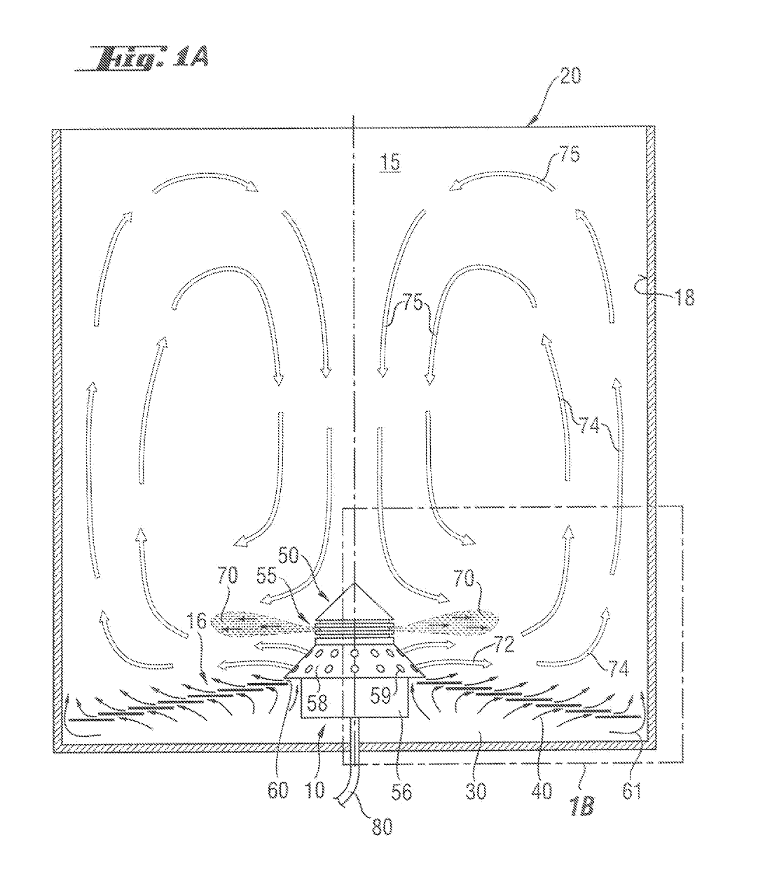

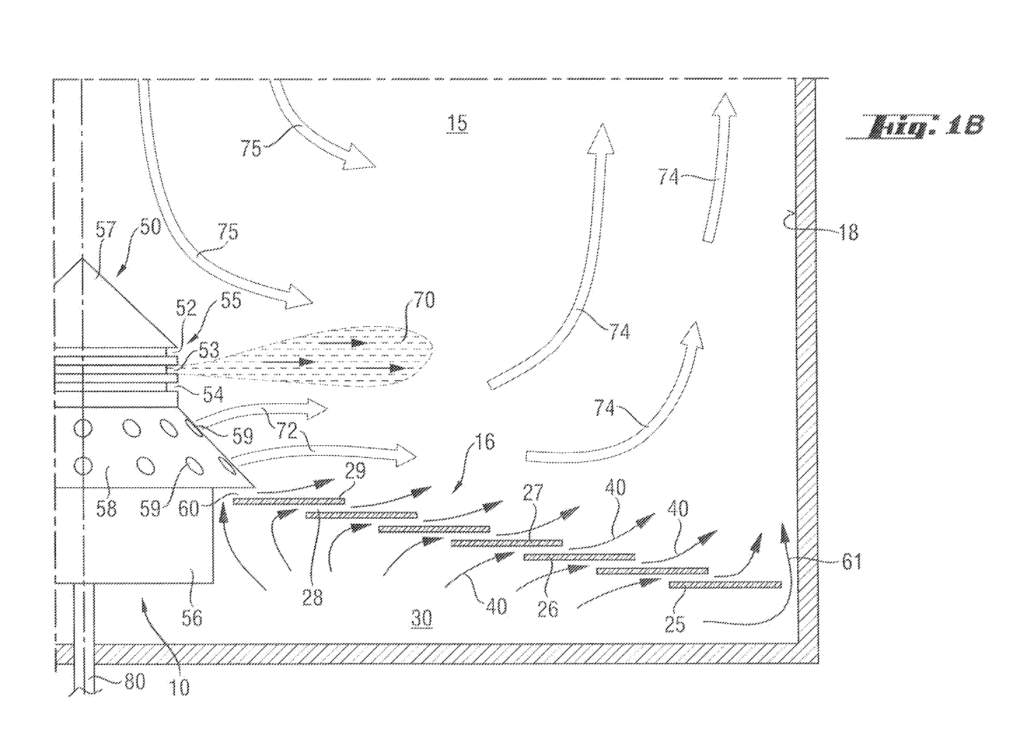

FIG. 1A: a vertical sectional view of a preferred device for carrying out the process according to the invention;

FIG. 1B an enlargement of the area framed in FIG. 1A numbered 1B;

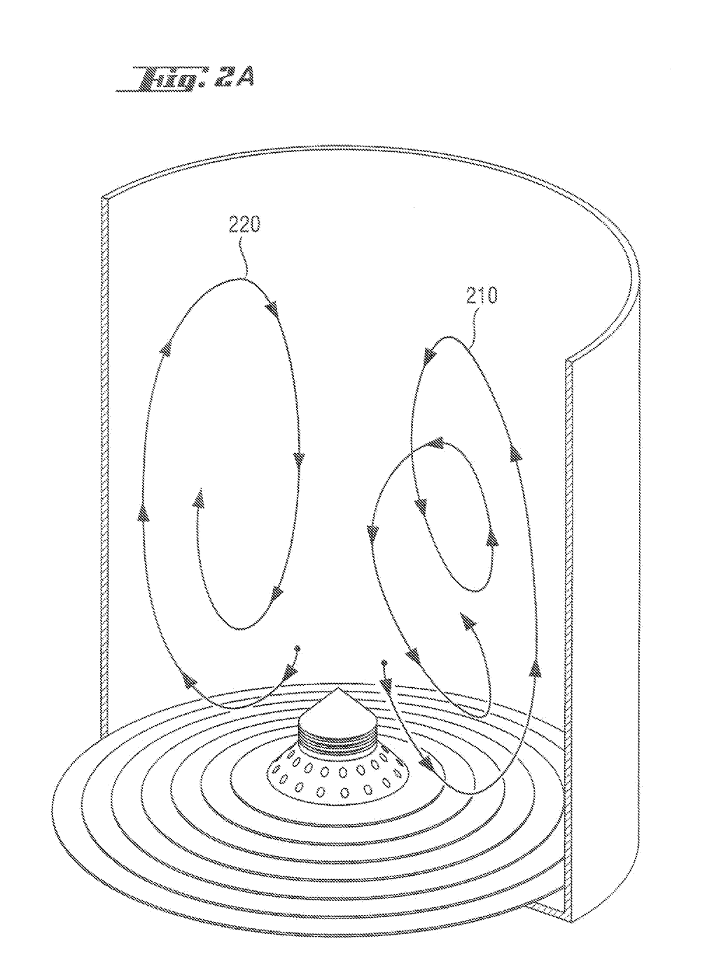

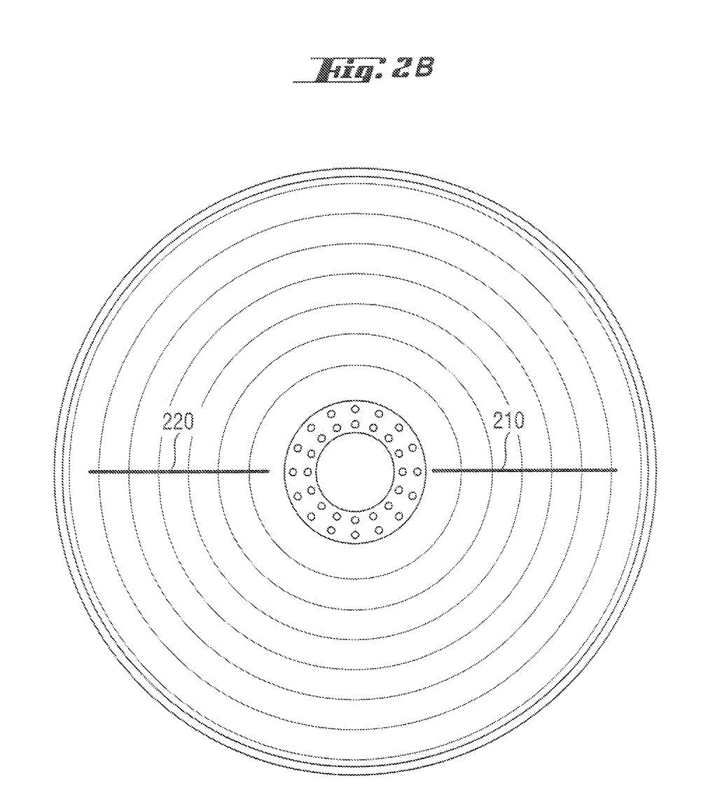

FIG. 2A a perspective sectional view of the preferred device, in which the movement paths of two elliptically circulating catalyst supports are represented schematically;

FIG. 2B a plan view of the preferred device and the movement paths according to FIG. 2A;

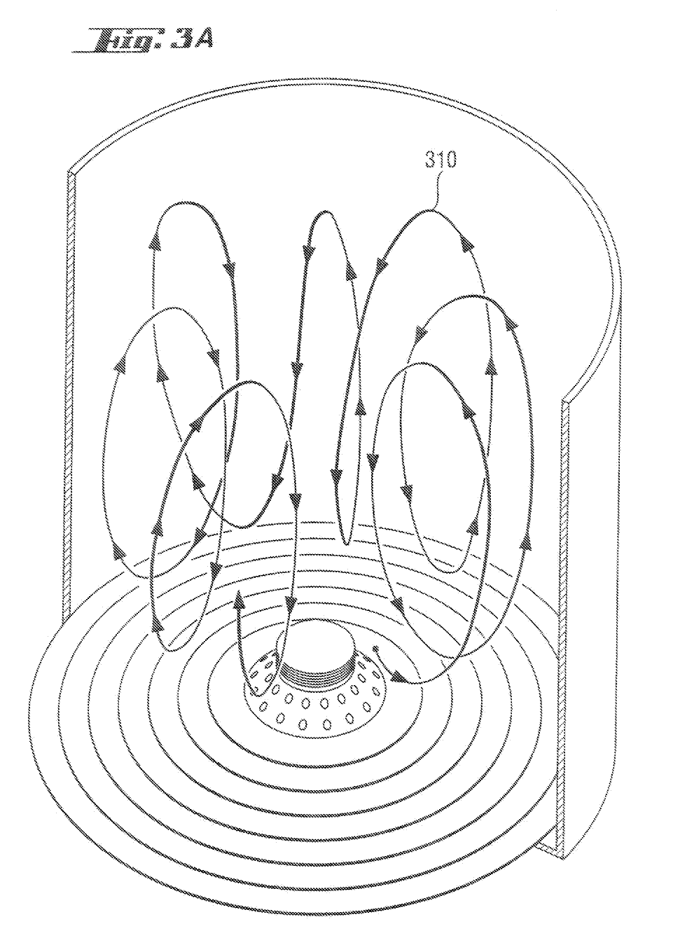

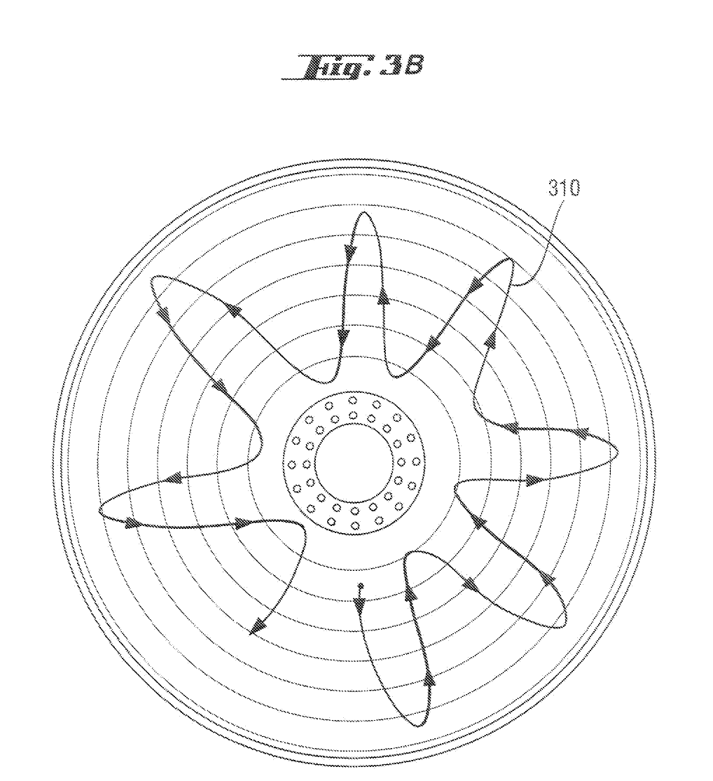

FIG. 3A a perspective sectional view of the preferred device, in which the movement path of a toroidally circulating catalyst support is represented schematically;

FIG. 3B a plan view of the preferred device and the movement path according to FIG. 3A.

A device, numbered 10 as a whole, for carrying out the process according to the invention is shown in FIG. 1A.

The device 10 has a container 20 with an upright cylindrical side wall 18 which encircles a process chamber 15.

The process chamber 15 has a bottom 16 below which is a blowing chamber 30.

The bottom 16 consists of a total of seven annular plates, laid one over the other, as guide plates. The seven annular plates are positioned one over the other in such a way that an outermost annular plate 25 forms an undermost annular plate on which the other six inner annular plates, each one partially overlapping the one beneath it, are placed.

For the sake of clarity, only some of the total of seven annular plates have reference numbers, for example the two overlapping annular plates 26 and 27. Due to this overlapping and spacing, an annular slot 28 is formed in each case between two annular plates, through which process air 40 can pass as a gas, with a predominantly horizontally aligned movement component, through the bottom 16.

An annular gap nozzle 50 is fitted from below in the central aperture of the central uppermost inner annular plate 29. The annular gap nozzle 50 has a mouth 55 which has a total of three orifice gaps 52, 53 and 54. All three orifice gaps 52, 53 and 54 are aligned so as to spray approximately parallel to the bottom 16, thus approximately horizontally, covering an angle of 360°. Alternatively, the spraying nozzle can be designed in such a way that the spraying cone runs diagonally upwards. Spray air is expressed as spray gas via the upper gap 52 and the lower gap 54, the solution to be sprayed is expressed through the central gap 53.

The annular gap nozzle 50 has a rod-shaped body 56 which extends downwards and contains the corresponding channels and feed lines which are known per se and therefore not represented in the drawing. The annular gap nozzle 50 can be formed for example with a so-called rotating annular gap, in which walls of the channels through which the solution is sprayed rotate relative to each other, in order to avoid blockages of the nozzle, thus making possible a uniform spraying out from the gap 53 over the whole 360°.

The annular gap nozzle 50 has a cone-shaped head 57 above the orifice gap 52.

In the area below the orifice gap 54 is a truncated-cone-shaped wall 58 which has numerous apertures 59. As can be seen from FIG. 1B, the underside of the truncated cone-shaped wall 58 rests on the innermost annular plate 29 in such a way that a slot 60 is formed, through which process air 40 can pass, between the underside of the truncated cone-shaped wall 58 and the annular plate 29 lying below and partially overlapping it.

The outer ring 25 is at a distance from the wall 18, with the result that process air 40 can enter the process chamber 15, with a predominantly vertical component, in the direction of the arrow given the reference number 61 and thereby gives the process air 40 entering the process chamber 15 through the slot 28 a component directed relatively sharply upwards.

The right-hand half of FIG. 1A shows what relationships form in the device 10 after entry.

A spray cloud 70 of the solution, the horizontal mirror plane of which runs roughly parallel to the bottom plane, emerges from the orifice gap 53. Air passing through the apertures 59 in the truncated cone-shaped wall 58, which can be for example process air 40, forms a supporting air flow 72 on the underside of the spray cloud 70. A radial flow in the direction of the wall 18 by which the process air 40 is deflected upwards, as represented by the arrow given the reference number 74, is formed by the process air 40 passing through the numerous slots 28. The catalyst supports are guided upwards by the deflected process air 40 in the area of the wall 18. The process air 40 and the catalyst supports to be treated then separate from each other, wherein the process air 40 is discharged through outlets, while the catalyst supports move radially inwards as shown by the arrow 75 and travel vertically downwards as a result of gravity in the direction of the conical head 57 of the annular gap nozzle 50. The descending catalyst supports are deflected there, carried to the upperside of the spray cloud 70 and treated there with the sprayed medium. The sprayed catalyst supports then move again towards the wall 18 and away from each other in the process, as a much larger space is available at the annular orifice gap 53 after the spray cloud 70 has left. In the area of the spray cloud 70, the catalyst supports to be treated encounter the sprayed solution and are moved in the direction of movement towards the wall 18, remaining apart from each other, and treated, i.e. dried, very uniformly and harmonically with the heated process air 40.

Two possible movement paths of two elliptically circulating catalyst supports are shown in FIG. 2A by means of the curve shapes given the reference numbers 210 and 220. The elliptical movement path 210 displays relatively large variations in the size of the major and minor axes compared with an ideal elliptical path. The elliptical movement path 220, on the other hand, displays relatively little variation in the size of the major and minor axes and describes close to an ideal elliptical path without a circumferential (horizontal) movement component, as can be seen from FIG. 2B.

A possible movement path of a toroidally circulating catalyst support is shown in FIG. 3A by means of the curve shape given the reference number 310. The toroidally running movement path 310 describes a section of the surface from a virtually uniform torus, the vertical cross-section of which is elliptical and the horizontal cross-section of which is annular. FIG. 3B shows the movement path 310 in plan view.

The following examples serve to illustrate the invention.

### EXAMPLE 1

70 g of a bentonite-based catalyst support from Süd-Chemie AG, Munich, Germany, with the trade name “KA-160” with the characteristics listed in Table 1:

TABLE 1

| Geometric form | sphere | |
|---|---|---|
| Diameter | 5 | mm |
| Moisture content | <2.0 | mass-% |
| Compressive strength | >60 | N |
| Bulk density | 554 | g l⁻¹ |
| Water absorbency | 62% | |
| Specific surface area (BET) | 158 | m² g⁻¹ |
| SiO₂ content | 93.2 | mass-% |
| Al2O₃ content | 2.2 | mass-% |
| Fe₂O₃ content | 0.35 | mass-% |
| TiO₂ content | (total) <1.5 | mass-% |
| MgO content | | |
| CaO content | | |
| K₂O content | | |
| Na₂O content | | |
| Loss on ignition 1000° C. | <0.3 | mass-% |
| Acidity | 53 | μval/g |
| BJH pore volume N₂ | 0.38 | cm³ g⁻¹ |

was converted into a fluidized-bed state, in which the catalyst supports circulated toroidally, by means of air temperature-controlled at 60° C. in a fluidized-bed reactor of the “Innojet® Aircoater 25” type from Innojet Technologies, Steinen, Germany.

The toroidally circulating catalyst supports were sprayed for a period of 1 h with 250 ml of an aqueous solution in which 10.63 g Ni(NO₃)₂, 0.31 g Mn(NO₃)₂ and 3.59 g Cu(NO₃)₂ were contained dissolved.

Once the solution had been deposited, the loaded catalyst supports were calcined at a temperature of 500° C. for a period of 2 h.

After the calcining, the shell catalysts had a total weight of 76.92 g. The proportion of Ni (as Ni metal) in the catalysts was 4.3 wt.-%, the proportion ot Mn (as Mn metal) 0.13 wt.-% and the proportion of Cu (as Cu metal) 1.6 wt.-%.

50 spheres were taken, halved and the layer thicknesses of a single half determined under the microscope at 4 points at 90° intervals. The layer thickness of a sphere corresponds to the average of the 4 measured values. The shell catalysts had an average shell thickness (arithmetic average of the 50 measured spheres) of 2161 μm.

### EXAMPLE 2

An experiment was carried out analogously to Example 1, except that the air to produce the fluidized bed was temperature-controlled at 70° C. and the toroidally circulating catalyst supports were sprayed with the aqueous solution for a period of 1.5 h.

After the calcining, the shell catalysts had a total weight of 76.69 g. The proportion of Ni, Mn and Cu (each as metal) in the catalysts was 4.1 wt.-%, 0.13 wt.-% and 1.33 wt.-% respectively. The catalysts had an average shell thickness of 914 μm.

### EXAMPLE 3

An experiment was carried out analogously to Example 1, except that the air to produce the fluidized bed was temperature-controlled at 80° C., the toroidally circulating catalyst support was sprayed with the aqueous solution for a period of 1.5 h and the concentration of manganese nitrate in the solution was three times higher.

After the calcining, the shell catalysts had a total weight of 75.08 g. The proportion of Ni, Mn and Cu (each as metal) in the catalysts was 4.0 wt.-%, 0.37 wt.-% and 1.3 wt.-% respectively. The shell catalysts had an average shell thickness of 482 μm.

It is to be noted that the shell thickness of the resulting catalysts can be set over a wide range via the processing temperature when spraying the catalyst supports with the solution.

The shell catalysts resulting from the process according to the invention have an exceptionally uniform shell thickness.

1. Shell catalyst comprising an open-pored catalyst support with a shell in which Ni and Cu and/or Pd and also in addition Mn and/or Mo are contained or in which Ni and also Mn and Mo are contained.

2. Shell catalyst according to claim 1, characterized in that Ni and also Cu and/or Pd are present in the oxidation state 0.

3. Shell catalyst according to claim 1, characterized in that Mn and/or Mo are present in oxide form.

4. Shell catalyst according to claim 1, characterized in that Ni, Cu and Mn are contained in the shell.

5. Shell catalyst according to claim 4, characterized in that the Ni/Cu atomic ratio of the shell catalyst is 0.5 to 30 and independently thereof the Ni/Mn atomic ratio 1 to 100.

6. Shell catalyst according to claim 1, characterized in that Ni, Pd and Mo are contained in the shell.

7. Shell catalyst according to claim 6, characterized in that the Ni/Pd atomic ratio of the shell catalyst is 10 to 500 and independently thereof the Ni/Mo atomic ratio 1 to 100.

8. Shell catalyst according to claim 1, characterized in that the proportion of Ni in the shell catalyst is 5 wt.-% to 15 wt.-%.

9. Shell catalyst according to claim 1, characterized in that the thickness of the shell is less than 2200 μm.

10. Shell catalyst according to claim 1, characterized in that the catalyst support has a specific surface area of 40 m²/g to 160 m²/g.

11. Shell catalyst according to claim 1, characterized in that the catalyst support has an integral pore volume according to BJH greater than/equal to 0.30 ml/g.

12. Shell catalyst according to claim 1, characterized in that the catalyst support has an integral pore volume according to BJH of 0.3 ml/g to 1.2 ml/g.

13. Shell catalyst according to claim 1, characterized in that at least 80% of the integral pore volume of the catalyst support is formed from mesopores and macropores.

14. Shell catalyst according to claim 1, characterized in that the bulk density of the catalyst support is more than 0.30 g/ml.

15. Shell catalyst according to claim 1, characterized in that the catalyst support has an average pore diameter of 8 nm to 50 nm.

16. Shell catalyst according to claim 1, characterized in that the catalyst support has an acidity of 1 μval/g to 150 μval/g.

17. Shell catalyst according to claim 1, characterized in that the catalyst support is formed as a shaped body.

18. Shell catalyst according to claim 17, characterized in that the catalyst support is formed as a sphere with a diameter of 1 mm to 25 mm.

19. Shell catalyst according to claim 1, characterized in that the catalyst support consists at least 50 wt.-% of SiO₂, Al₂O₃, an aluminum silicate, ZrO₂, TiO₂, HfO₂, MgO, niobium oxide or a natural sheet silicate or at least 50 wt.-% of a mixture of two or more of the above-mentioned materials.

20. Shell catalyst according to claim 1, characterized in that the catalyst support consists at least 80 wt.-% of a natural sheet silicate.

21. Shell catalyst according to claim 1, characterized in that the catalyst support consists at least 80 wt.-% of montmorillonite.

22. Shell catalyst according to claim 1, characterized in that the catalyst has a hardness greater than/equal to 20 N.

23. Shell catalyst according to claim 1, characterized in that the catalyst comprises at least one promoter selected from the group consisting of P, Na, K, Co and Mg.

24. Process in which a catalyst support is sprayed with a solution in which a Ni compound and also a Cu compound and/or a Pd compound and also furthermore a Mn compound and/or a Mo compound are contained dissolved or in which a Ni compound and also a Mn compound and a Mo compound are contained dissolved.

25. Process according to claim 24, comprising
generating a fluidized bed of catalyst supports by means of a gas, whereas the catalyst supports move in the fluidized bed on an elliptical or toroidal path;
spraying of the catalyst supports moving in the fluidized bed on an elliptical or toroidal path with the solution.

26. Process according to claim 25, characterized in that the catalyst supports move in the fluidized bed on a toroidal path.

27. Process according to claim 24, characterized in that the process furthermore comprises
drying of the catalyst supports sprayed with the solution.

28. Process according to claim 24, characterized in that the process furthermore comprises
calcining of the catalyst supports sprayed with the solution at a temperature at which the metal component of the metal compounds sprayed onto the catalyst supports is converted into an oxide form.

29. Process according to claim 24, characterized in that the process furthermore comprises
converting the metal component of one or more metal compounds sprayed onto the catalyst support into the oxidation state 0.

30. Process according to claim 24, characterized in that a Ni compound, a Cu compound and a Mn compound are contained dissolved in the solution.

31. Process according to claim 24, characterized in that a Ni compound, a Pd compound and a Mo compound are contained dissolved in the solution.

32. Process according to claim 24, characterized in that the metal compounds contained in the solution are halogen-free metal compounds.

33. Process according to claim 24, characterized in that the metal compounds contained in the solution are nitrate compounds of the respective metals.

34. Process according to claim 24, characterized in that the solution is an aqueous solution.

35. Process according to claim 24, characterized in that the process is carried out using a device which is set up to produce a fluidized bed of a particulate material by means of a gas, wherein the particles of the material move in the fluidized bed on an elliptical or toroidal path.

36. Process according to claim 35, characterized in that the device comprises a process chamber with a bottom and a side wall, wherein the gas is fed into the process chamber with a horizontal movement component aligned radially outwards through the bottom of the process chamber whereas the bottom is preferably constructed of several overlapping annular guide plates laid one over the other between which annular slots are formed, in order to produce the catalyst support fluidized bed.

37. Process according to claim 36, characterized in that a circumferential flow component is imposed on the gas fed into the process chamber.

38. Process according to claim 37, characterized in that the circumferential flow component is imposed on the gas fed into the process chamber by means of guide elements which are arranged between the annular guide plates.

39. Process according to claim 37, characterized in that the circumferential flow component is imposed on the gas fed into the process chamber by feeding additional gas through the bottom of the process chamber into the process chamber with a movement component aligned diagonally upwards.

40. Process according to claim 36, characterized in that the catalyst supports moving in the fluidized bed on an elliptical or toroidal path are sprayed with the solution
by means of an annular gap nozzle which atomizes a spray cloud which runs substantially parallel to the plane of the bottom.

41. Process according to claim 40, characterized in that the annular gap nozzle is arranged centrally on the bottom and the mouth of the annular gap nozzle is embedded in the fluidized bed.

42. Process according to claim 40, characterized in that a gas support cushion is produced on the underside of the spray cloud.

43. Process according to claim 25, characterized in that the gas is selected from the group consisting of air, oxygen, nitrogen and the noble gases and also mixtures of the above gases.

44. Process according to claim 24, characterized in that the process is carried out at a temperature greater than/equal to 60° C.

45. Process according to claim 36, characterized in that the gas is enriched with the solvent of the solution before feeding into the process chamber.

46. Use of a shell catalyst according to claim 1 for the hydrogenation of aromatics, alkines, olefins, aldehydes and oxo products.
